SEQUENCE LISTING (45 sequences)

SEQ ID NO: 1 - protein, 11 aa, synthetic construct
GPAPELLGGGS

SEQ ID NO: 2 - protein, 11 aa, synthetic construct
GPAPLGLGGGS

SEQ ID NO: 3 - protein, 12 aa, synthetic construct
GPAPELLGGGGS

SEQ ID NO: 4 - protein, 12 aa, synthetic construct
GPAPLGLGGGGS

SEQ ID NO: 5 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 6 - protein, 330 aa, Homo sapiens [sequence omitted]

SEQ ID NO: 7 - protein, 107 aa, Homo sapiens [sequence omitted]

SEQ ID NO: 8 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 10 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 5 aa, synthetic construct
TYAIS

SEQ ID NO: 13 - protein, 17 aa, synthetic construct
GIIPIFGKAHYAQKFQG

SEQ ID NO: 14 - protein, 14 aa, synthetic construct
KFHFVSGSPFGMDV

SEQ ID NO: 15 - protein, 11 aa, synthetic construct
RASQSVSSYLA

SEQ ID NO: 16 - protein, 7 aa, synthetic construct
DASNRAT

SEQ ID NO: 17 - protein, 8 aa, synthetic construct
QQRSNWPT

SEQ ID NO: 18 - protein, 5 aa, synthetic construct
SYVLH

SEQ ID NO: 19 - protein, 17 aa, synthetic construct
VISDDGRNKYFADSVKG

SEQ ID NO: 20 - protein, 10 aa, synthetic construct
EGYSGSWFDY

SEQ ID NO: 21 - protein, 11 aa, synthetic construct
RASQGISSALA

SEQ ID NO: 22 - protein, 7 aa, synthetic construct
GASSLEG

SEQ ID NO: 23 - protein, 9 aa, synthetic construct
QQFNSYPFT

SEQ ID NO: 24 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 25 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 26 - protein, 119 aa, synthetic construct
QVQLVESGGGVVQPGRSLRLSCAASGFTFSSYVLHWVRQAPGKGLDWVAVISDDGRNKYFADSVKGRFTISRDNSKNTLYLQMNSLRAEDTAVYYCVREGYSGSWFDYWGQGTLVTVSS

SEQ ID NO: 27 - protein, 107 aa, synthetic construct
AIQLTQSPSSLSASVGDRVTITCRASQGISSALAWYQQKPGKAPKLLIYGASSLEGGVPSRFSGSGSGTDFTLTISSLQPEDFATYYCQQFNSYPFTFGPGTKVDIK

SEQ ID NO: 28 - protein, 438 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSAIQLTQSPSSLSASVGDRVTITCRASQGISSALAWYQQKPGKAPKLLIYGASSLEGGVPSRFSGSGSGTDFTLTISSLQPEDFATYYCQQFNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 439 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGGSAIQLTQSPSSLSASVGDRVTITCRASQGISSALAWYQQKPGKAPKLLIYGASSLEGGVPSRFSGSGSGTDFTLTISSLQPEDFATYYCQQFNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 686 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLVESGGGVVQPGRSLRLSCAASGFTFSSYVLHWVRQAPGKGLDWVAVISDDGRNKYFADSVKGRFTISRDNSKNTLYLQMNSLRAEDTAVYYCVREGYSGSWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 687 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGGSQVQLVESGGGVVQPGRSLRLSCAASGFTFSSYVLHWVRQAPGKGLDWVAVISDDGRNKYFADSVKGRFTISRDNSKNTLYLQMNSLRAEDTAVYYCVREGYSGSWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 223 aa, synthetic construct
SCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - skipped

SEQ ID NO: 34 - protein, 7 aa, synthetic construct
GDTFSTY

SEQ ID NO: 35 - protein, 6 aa, synthetic construct
IPIFGK

SEQ ID NO: 36 - protein, 8 aa, synthetic construct
GDTFSTYA

SEQ ID NO: 37 - protein, 8 aa, synthetic construct
IIPIFGKA

SEQ ID NO: 38 - protein, 16 aa, synthetic construct
ARKFHFVSGSPFGMDV

SEQ ID NO: 39 - protein, 6 aa, synthetic construct
QSVSSY

SEQ ID NO: 40 - protein, 7 aa, synthetic construct
GFTFSSY

SEQ ID NO: 41 - protein, 6 aa, synthetic construct
SDDGRN

SEQ ID NO: 42 - protein, 8 aa, synthetic construct
GFTFSSYV

SEQ ID NO: 43 - protein, 8 aa, synthetic construct
ISDDGRNK

SEQ ID NO: 44 - protein, 12 aa, synthetic construct
VREGYSGSWFDY

SEQ ID NO: 45 - protein, 6 aa, synthetic construct
QGISSA

(19) United States
(12) Patent Application Publication
(10) Pub. No.: US 2026/0132204 A1
(43) Pub. Date: May 14, 2026

(54) ACTIVATABLE BISPECIFIC ANTI-CD89 AND ANTI-PD-L1 PROTEINS AND USES THEREOF

(71) Applicant: CENTESSA PHARMACEUTICALS (UK) LIMITED, Altrincham (GB)

(72) Inventor: William James Jonathan FINLAY, Glasgow (GB)

(21) Appl. No.: 19/360,170

(22) Filed: Oct. 16, 2025

Related U.S. Application Data
(63) Continuation of application No. PCT/GB2024/051028, filed on Apr. 19, 2024.
(60) Provisional application No. 63/497,092, filed on Apr. 19, 2023.

(51) Int. Cl. C07K 16/28

(52) U.S. Cl. CPC C07K 16/283; C07K 16/2827; C07K 2317/24; C07K 2317/31; C07K 2317/52

(57) ABSTRACT

Provided herein are protein molecules that specifically bind PD-L1 and also exhibit activatable specific CD89 binding in diseased tissues. Further provided herein are uses of such protein molecules to treat cancer.

### CROSS-REFERENCE

This application is a continuation application of International Application No. PCT/GB2024/051028, filed on Apr. 19, 2024, which claims the benefit of U.S. Provisional Application No. 63/497,092, filed on Apr. 19, 2023, each of which is incorporated herein by reference in its entirety.

### SEQUENCE LISTING

The instant application contains a Sequence Listing which has been submitted electronically in XML format and is hereby incorporated by reference in its entirety. Said XML copy, created on Apr. 19, 2024, is named P90092.WO01_ST.26_Sequence_Listing.xml and is 45,056 bytes in size.

### TECHNICAL FIELD

The disclosure relates to activatable bispecific proteins and treatments for cancer.

### BACKGROUND

In immune oncology therapy, few of the key drug targets are exclusively expressed in diseased tissue, with the majority also being expressed in non-diseased tissue. In addition, many drugs employed in cancer treatment employ highly potent cell-killing mechanisms of action. As a result, engagement of the target by the drug in non-diseased tissue often causes unwanted side effects.

PD-L1 is a cell surface receptor that is a member of the immunoglobulin superfamily and is principally expressed on myeloid cells and regulatory T (Treg) cells in non-diseased tissues. However, PD-L1 has also been observed to be highly expressed on some cancer cells. PD-L1 binds to the membrane protein PD1. The interaction of PD-L1 with PD1 on T cells down-regulates T cell inflammatory activity, which promotes immune self-tolerance. PD-L1 is, therefore, described as an immune checkpoint. Hence, antagonistic anti-PD-L1 monoclonal antibodies that block interaction with PD1 have demonstrated the potential to act as well-tolerated immunotherapeutic agents in disease settings such as cancer, by liberating anti-cancer T cell responses from natural immune restriction. As such, PD-L1 is a drug target used to amplify the adaptive immune system's anti-cancer effects. In many cases, however, tumors may use multiple “escape mechanisms” to negate the effects of anti-PD-L1 agents.

CD89 is the receptor for IgA and binds the heavy-chain constant region of IgA antibodies. CD89 is present on the cell surface of myeloid lineage cells, including neutrophils, monocytes, macrophages, and eosinophils. Binding and activation of CD89 by IgA provides a powerful induction of antibody-dependent cellular cytotoxicity (ADCC) and antibody-dependent cellular phagocytosis (ADCP) against IgA-opsonized cells that can be significantly more powerful than the induction induced by a similar IgG. Despite these observations, the development of IgA antibodies as therapeutic agents has been generally unsuccessful as IgA antibody structures are difficult to express and purify, and suffer from poor pharmacokinetics (PK) in humans that minimizes exposure in diseased tissues.

Historically, antibodies have been generated against the CD89 extracellular domain which do not compete for binding with endogenous IgA. Upon CD89 binding, these antibodies can induce a direct activation signal that leads to increased inflammatory activity of innate immune cells. Using binding domains from these antibodies, CD89-ligating bispecific agents have also been generated that may direct innate immune cell killing activity to target-enriched diseased tissues. Anti-PD-L1 antibodies could therefore be made more potent and broadly acting therapeutic agents by gaining the ability to also bind CD89 and thereby strongly enhance innate immune cell killing mechanisms in PD-L1 antibody-resistant disease settings. This would combine a key checkpoint inhibitor function with an inducible “synthetic IgA immunity” that can synergistically stimulate the adaptive immune system. However, the ability to make this combination work in a single therapeutic agent structure (e.g., in standard bispecific antibody format with fully active PD-L1 and CD89 binding domains) is limited by the relatively broad expression profiles of both PD-L1 and CD89 on many cell types such as T cells and innate immune cells, as well as others. This broad expression profile may lead not only to dose-limiting toxicities for PD-L1/CD89 binding agents, but also to profound peripheral sink/biodistribution problems that limit the ability of such agents to achieve high enough exposure in diseased tissues to exploit the combined mechanisms. There is, therefore, a need for engineered forms of bispecific binding proteins with activity specifically targeted to the diseased tissue environment.

### SUMMARY

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD89 VH domain, and a second CH1 domain; wherein the light chain comprises, in N-terminus to C-terminus order, an anti-PD-L1 light chain variable (VL) domain, a first immunoglobulin light chain constant region, a second linker, an anti-CD89 VL domain, and a second immunoglobulin light chain constant region.

In some embodiments, the heavy chain comprises in N-terminus to C-terminus order, the anti-PD-L1 VH domain, the first CH1 domain, the first linker, the anti-CD89 VH domain, the second CH1 domain, a hinge, a CH2 domain, and a CH3 domain.

In some embodiments, the protein further comprises a third polypeptide chain comprising a hinge and a Fc region. In some embodiments, the third polypeptide chain comprises the amino acid sequence of SEQ ID NO: 32.

In some embodiments, the first linker comprises the amino acid sequence of SEQ ID NO: 1, SEQ ID NO: 2, SEQ ID NO: 3, or SEQ ID NO: 4. In some embodiments, the second linker comprises the amino acid sequence of SEQ ID NO: 1, SEQ ID NO: 2, SEQ ID NO: 3, or SEQ ID NO: 4.

In some embodiments, the anti-PD-L1 VH domain comprises a HCDR1 comprising the amino acid sequence of SEQ ID NO: 12, a HCDR2 comprising the amino acid sequence of SEQ ID NO: 13, and a HCDR3 comprising the amino acid sequence of SEQ ID NO: 14; the anti-PD-L1 VL domain comprises a LCDR1 comprising the amino acid sequence of SEQ ID NO: 15, a LCDR2 comprising the amino acid sequence of SEQ ID NO: 16, and a LCDR3 comprising the amino acid sequence of SEQ ID NO: 17; the anti-CD89 VH domain comprises a HCDR1 comprising the amino acid sequence of SEQ ID NO: 18, a HCDR2 comprising the amino acid sequence of SEQ ID NO: 19, and a HCDR3 comprising the amino acid sequence of SEQ ID NO: 20; and the anti-CD89 VL domain comprises a LCDR1 comprising the amino acid sequence of SEQ ID NO: 21, a LCDR2 comprising the amino acid sequence of SEQ ID NO: 22, and a LCDR3 comprising the amino acid sequence of SEQ ID NO: 23.

In some embodiments, the anti-PD-L1 VH domain comprises the amino acid sequence of SEQ ID NO: 24, and the anti-PD-L1 VL domain comprises the amino acid sequence of SEQ ID NO: 25. In some embodiments, the anti-CD89 VH domain comprises the amino acid sequence of SEQ ID NO: 26. In some embodiments, the anti-CD89 VL domain comprises the amino acid sequence of SEQ ID NO: 27.

In some embodiments, the heavy chain comprises the amino acid sequence of SEQ ID NO: 30 or SEQ ID NO: 31. In some embodiments, the light chain comprises the amino acid sequence of SEQ ID NO: 28 or SEQ ID NO: 29.

Provided herein is a protein wherein
(a) the heavy chain comprises the amino acid sequence of SEQ ID NO: 30, the light chain comprises the amino acid sequence of SEQ ID NO: 28, and the third polypeptide chain comprises the amino acid sequence of SEQ ID NO: 32; or
(b) the heavy chain comprises the amino acid sequence of SEQ ID NO: 31, the light chain comprises the amino acid sequence of SEQ ID NO: 29, and the third polypeptide chain comprises the amino acid sequence of SEQ ID NO: 32.

In some embodiments, the heavy chain comprises an IgG, IgE, IgM, IgD, IgA, or IgY constant region. In some embodiments, the heavy chain comprises an IgG1, IgG2, IgG3, IgG4, IgA1 or IgA2 constant region. In some embodiments, the heavy chain comprises an immunologically inert constant region. In some embodiments, the heavy chain comprises a wild-type human IgG1 constant region, a human IgG1 constant region comprising the amino acid substitutions L234A, L235A and G237A, a wild-type human IgG2 constant region, a wild-type human IgG4 constant region, or a human IgG4 constant region comprising the amino acid substitution S228P, wherein numbering is according to the EU index as in Kabat.

Provided herein is an immunoconjugate comprising a protein disclosed herein, linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Provided herein is a pharmaceutical composition comprising a protein or an immunoconjugate disclosed herein, and a pharmaceutically acceptable carrier, diluent or excipient.

Provided herein is a nucleic acid molecule encoding (a) the heavy chain amino acid sequence; (b) the light chain amino acid sequence; or (c) both the heavy chain and the light chain amino acid sequences of the protein disclosed herein.

Provided herein is an expression vector comprising a nucleic acid molecule disclosed herein.

Provided herein is a recombinant host cell comprising a nucleic acid molecule or an expression vector disclosed herein.

Provided herein is a method of producing a protein, the method comprising: culturing a recombinant host cell comprising an expression vector disclosed herein under conditions whereby the nucleic acid molecule is expressed, thereby producing the protein; and isolating the protein from the host cell or culture.

Provided herein is a method for enhancing an anti-cancer immune response in a subject, the method comprising administering to the subject a therapeutically effective amount of a protein, an immunoconjugate, or a pharmaceutical composition disclosed herein.

Provided herein is a method for treating cancer in a subject, the method comprising administering to the subject a therapeutically effective amount of a protein, an immunoconjugate, or a pharmaceutical composition disclosed herein.

Provided herein is a method for ameliorating a symptom of cancer in a subject, the method comprising administering to the subject a therapeutically effective amount of a protein, an immunoconjugate, or a pharmaceutical composition disclosed herein.

In some embodiments of the methods provided herein, the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

### BRIEF DESCRIPTION OF THE DRAWINGS

FIG. 1 depicts a diagram of an exemplary asymmetric one-arm construct of the protein molecule disclosed herein in intact (left), activated protease-cleaved (middle) and deactivated protease-cleaved (right) conformations. In an intact conformation, the anti-PD-L1 Fab binding domain is exposed and able to bind their cognate target. The anti-CD89 Fab domain is inhibited from binding by linkers in both the heavy and light chains that are both proteolytically cleavable and may be sequentially cleaved by matrix metalloproteases (MMPs) and/or cathepsins. A first cleavage event creates an intermediate active state which allows both anti-PD-L1 and anti-CD89 Fabs from a single protein construct to bind their cognate targets, thereby potentially directing activation of CD89+ immune cells in the PD-L1+ immunosuppressive microenvironment. A second cleavage dissociates the anti-PD-L1 and anti-CD89 Fabs, removing the ability of a single molecule to bind both PD-L1 and CD89 simultaneously.

FIG. 2 depicts a diagram of the mechanism of activity of activatable bispecific protein molecules provided herein. As the liberated activity of the CD89 binding domain is monomeric in nature, it cannot cause CD89 activation directly and relies upon binding to PD-L1+ cells, leading to presentation of CD89 binding in trans to nearby immune cells to induce CD89 signaling. The activated molecule thereby simultaneously blocks PD-L1/PD1 signaling to reinstate uninhibited TCR activation in T cells and provides CD89 signaling to amplify innate immune cell ADCC and ADCP activity.

FIG. 3 depicts the structure of an exemplary symmetric two-arm construct of the protein molecule described herein.

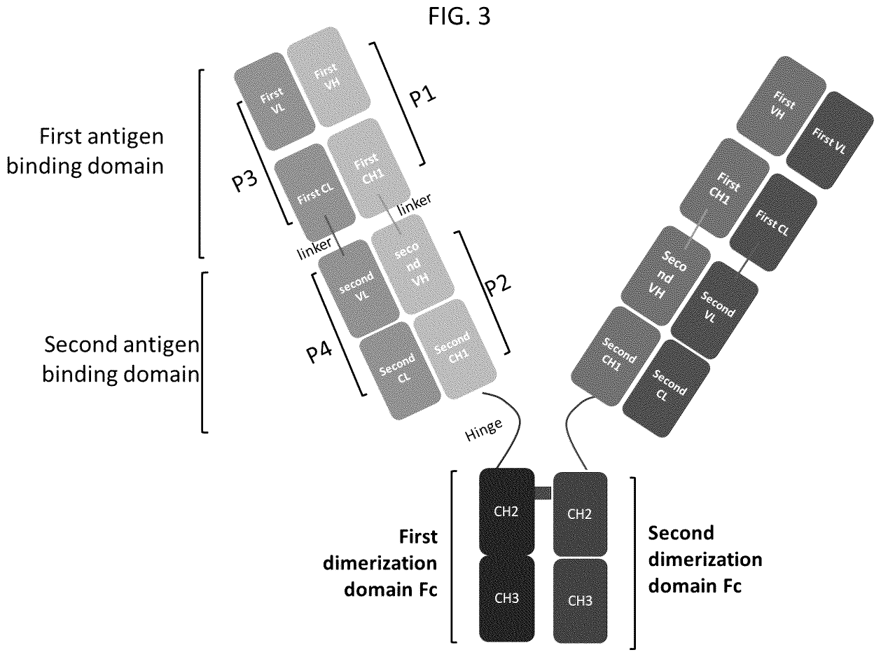

### DETAILED DESCRIPTION

There are two key issues that restrict the efficacy in CD89-activating, tumor-targeting bispecific antibody drugs for oncology:
1) That the antibody target protein found on cancer cells (e.g., PD-L1) is potentially expressed on many different cell classes in the body, not just on the tumor cell. This off-tumor target expression may lead to dose-limiting side effect risks as the CD89 binding domain in a standard bispecific molecule is constitutively active and may therefore direct CD89+ cell activation in the presence of any PD-L1+ cell, whether it is in diseased tissue or not. Off-tumor target expression may also lead to antigen “sink” effects, which reduces the amount of drug penetrating the tumor.
2) CD89-positive cells such as neutrophils and monocytes are found in the bloodstream and at high concentration in other tissues, creating a large sink effect for this arm, affecting biodistribution and free drug availability for tumor penetration.

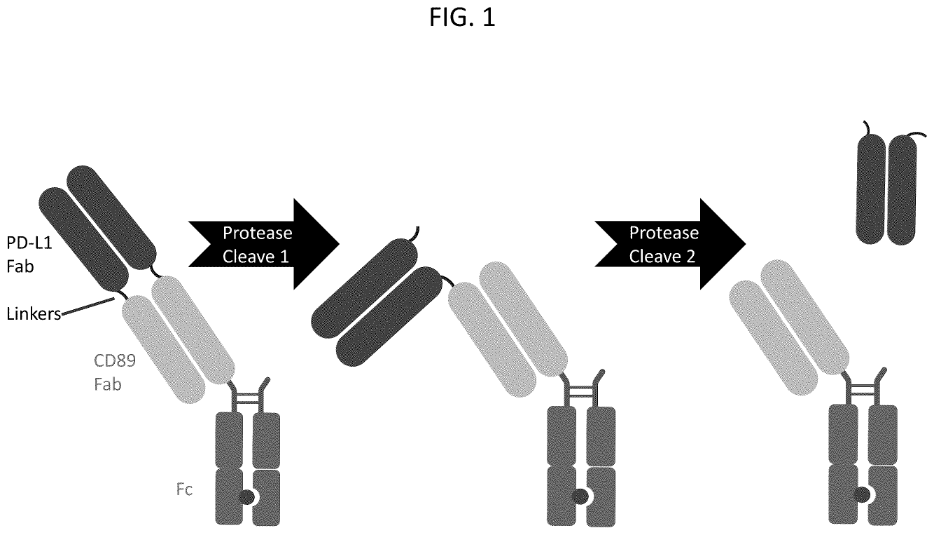

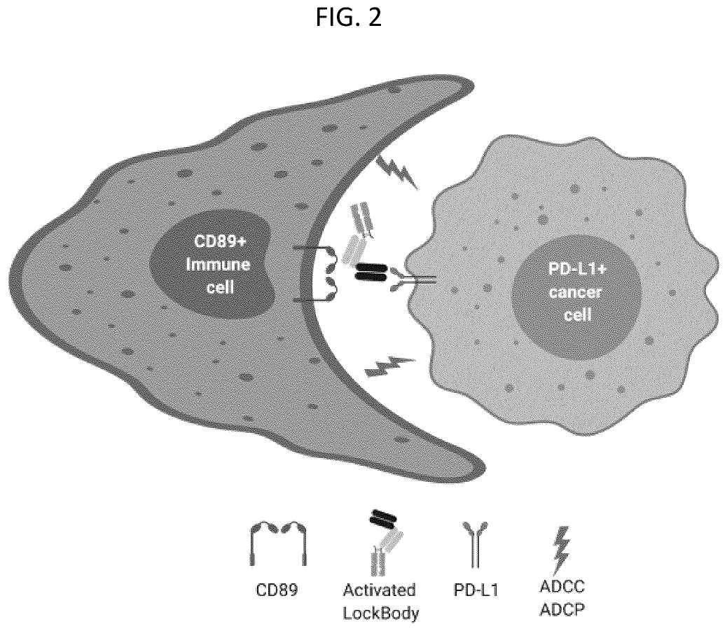

Both factors described above minimize the potential safety and efficacy of bispecific antibodies that drive CD89 engagement and activation. The anti-PD-L1 and anti-CD89 proteins (see, e.g., FIG. 1) provided herein overcome the peripheral sink and toxicity issues by minimizing binding of CD89 outside of diseased tissue. This effect is achieved by adding PD-L1 binding domains and linkers above (i.e., amino-terminal to) the CD89 binding domains. The use of appropriate upper domain and linker combinations results in a configuration that minimizes binding activity in the lower (i.e., carboxy-terminal) CD89 domain. The PD-L1 domain then drives high concentration in PD-L1 enriched tumor microenvironments. The protein construct linker system exploits the elevated MMP and cathepsin activity that is common in solid tumors to cleave the linker peptides, exposing the CD89 binding domains and thereby conditionally activating the CD89-activating activity in the tumor, rather than the periphery. These combined biological functions thereby afford the molecule the potential to avoid the peripheral CD89 sink and maximize T cell immune responses to cancer cells, as outlined in FIG. 2.

Provided herein are proteins that are conditionally active in diseased human tissues. The proteins of the disclosure are fully active in specifically binding and blocking PD-L1 throughout the body; exhibit minimized binding of CD89 in healthy tissue; and become highly activated in CD89 binding and activation once in the PD-L1-positive diseased tissue environment. A protein of the disclosure comprises a CD89 binding domain that is masked by a PD-L1 binding domain in non-diseased tissues. The protein also comprises two peptide linkers that are cleaved by one or more proteases expressed in a diseased tissue (e.g, a tumor). The linker cleavage unmasks the CD89 binding domain in the diseased tissue, thus allowing binding and/or function of the protein selectively in the diseased tissue.

### Protein Molecules

Provided herein are proteins comprising two Fab fragments (an anti-PD-L1 Fab and an anti-CD89 Fab). The protein is monovalent when in the intact structure and can only have a maximum of monovalent CD89 binding when activated, to minimize peripheral toxicity risk associated with bivalent, activating anti-CD89 antibodies.

In some embodiments, a protein comprises a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD89 VH domain, and a second CH1 domain; wherein the light chain comprises, in N-terminus to C-terminus order, an anti-PD-L1 light chain variable (VL) domain, a first immunoglobulin light chain constant region, a second linker, an anti-CD89 VL domain, and a second immunoglobulin light chain constant region. In some embodiments, the heavy chain further comprises an immunoglobulin hinge region and an Fc domain at its C-terminus. In some embodiments, the heavy chain comprises in N-terminus to C-terminus order, the anti-PD-L1 VH domain, the first CH1 domain, the first linker, the anti-CD89 VH domain, the second CH1 domain, a hinge, a CH2 domain, and a CH3 domain.

In some embodiments, the protein further comprises a third polypeptide chain comprising a hinge and a Fc region. The third polypeptide chain may be referred to as a “Fc-stump”.

A diagram of an illustrative protein of the disclosure, with labeled domains, is shown in FIG. 1.

The first linker and the second linker are cleavable by matrix metalloproteases (MMPs) and/or cathepsins found in diseased tissues, such as tumors. The linkers in the protein are immunoglobulin-derived hinge sequences that are both proteolytically sensitive and may be sequentially cleaved, with a first cleavage taking the intact structure and creating an intermediate active state which allows an anti-PD-L1 Fab and an anti-CD89 Fab from a single protein construct to bind their cognate targets. A second cleavage event in the second linker removes the covalent linkage between the anti-PD-L1 Fab and the anti-CD89 Fab, abrogating the ability of the molecule to recruit T cell killing of PD-L1+ cells. This secondary cleavage event thereby constitutes a “self-destruct mechanism” that minimizes risk of activated molecule escaping the tumor microenvironment. Cleaved linkers based on immunoglobulin hinge sequences may also recruit increased immune effector function (antibody-dependent cellular cytotoxicity (ADCC), complement-dependent cytotoxicity (CDC) and antibody-dependent cellular phagocytosis (ADCP)) at the cell membrane via endogenous anti-hinge antibodies, which are a known phenomenon in human patients with (and even without) underlying autoreactive disease.

In some embodiments, a protein comprises one or more amino acid sequences provided in Table 1 or Table 2.

TABLE 1
Exemplary sequences

| Domain or Region | Sequence | SEQ ID NO |
|---|---|---|
| Linker LHL | GPAPELLGGGS | 1 |
| Linker LHL-F | GPAPLGLGGGS | 2 |
| Linker LHLX | GPAPELLGGGGS | 3 |
| Linker LHLX-F | GPAPLGLGGGGS | 4 |
| Human CH1 domain | ASTKGPSVFPLAPSSKSTSGGTAALGCLVKDYFPEPVTV | 5 |
| | SWNSGALTSGVHTFPAVLQSSGLYSLSSVVTVPSSSLGT | |
| | QTYICNVNHKPSNTKVDKKVEPKSC | |
| Human CH1-hinge- | ASTKGPSVFPLAPSSKSTSGGTAALGCLVKDYFPEPVTV | 6 |
| CH2-CH3 sequence | SWNSGALTSGVHTFPAVLQSSGLYSLSSVVTVPSSSLGT | |
| (IgG1 wild type) | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLH | |
| | QDWLNGKEYKCKVSNKALPAPIEKTISKAKGQPREPQV | |
| | YTLPPSRDELTKNQVSLTCLVKGFYPSDIAVEWESNGQP | |
| | ENNYKTTPPVLDSDGSFFLYSKLTVDKSRWQQGNVFSC | |
| | SVMHEALHNHYTQKSLSLSPGK | |
| Human Cₖₐₚₚₐ domain | RTVAAPSVFIFPPSDEQLKSGTASVVCLLNNFYPREAKV | 7 |
| (Ck) | QWKVDNALQSGNSQESVTEQDSKDSTYSLSSTLTLSKA | |
| | DYEKHKVYACEVTHQGLSSPVTKSFNRGEC | |
| Human IgG1-3M | ASTKGPSVFPLAPSSKSTSGGTAALGCLVKDYFPEPVTV | 8 |
| | SWNSGALTSGVHTFPAVLQSSGLYSLSSVVTVPSSSLGT | |
| | QTYICNVNHKPSNTKVDKKVEPKSCDKTHTCPPCPAPEA | |
| | AGAPSVFLFPPKPKDTLMISRTPEVTCVVVDVSHEDPEV | |
| | KFNWYVDGVEVHNAKTKPREEQYNSTYRVVSVLTVLH | |
| | QDWLNGKEYKCKVSNKALPAPIEKTISKAKGQPREPQV | |
| | YTLPPSRDELTKNQVSLTCLVKGFYPSDIAVEWESNGQP | |
| | ENNYKTTPPVLDSDGSFFLYSKLTVDKSRWQQGNVFSC | |
| | SVMHEALHNHYTQKSLSLSPGK | |
| Human IgG2 wild | ASTKGPSVFPLAPCSRSTSESTAALGCLVKDYFPEPVTVS | 9 |
| type | WNSGALTSGVHTFPAVLQSSGLYSLSSVVTVPSSNFGTQ | |
| | TYTCNVDHKPSNTKVDKTVERKCCVECPPCPAPPVAGP | |
| | SVFLFPPKPKDTLMISRTPEVTCVVVDVSHEDPEVQFNW | |
| | YVDGVEVHNAKTKPREEQFNSTFRVVSVLTVVHQDWL | |
| | NGKEYKCKVSNKGLPAPIEKTISKTKGQPREPQVYTLPP | |
| | SREEMTKNQVSLTCLVKGFYPSDISVEWESNGQPENNY | |
| | KTTPPMLDSDGSFFLYSKLTVDKSRWQQGNVFSCSVMH | |
| | EALHNHYTQKSLSLSPGK | |
| Human IgG4 wild | ASTKGPSVFPLAPCSRSTSESTAALGCLVKDYFPEPVTVS | 10 |
| type | WNSGALTSGVHTFPAVLQSSGLYSLSSVVTVPSSSLGTK | |
| | TYTCNVDHKPSNTKVDKRVESKYGPPCPSCPAPEFLGGP | |
| | SVFLFPPKPKDTLMISRTPEVTCVVVDVSQEDPEVQFNW | |
| | YVDGVEVHNAKTKPREEQFNSTYRVVSVLTVLHQDWL | |
| | NGKEYKCKVSNKGLPSSIEKTISKAKGQPREPQVYTLPPS | |
| | QEEMTKNQVSLTCLVKGFYPSDIAVEWESNGQPENNYK | |
| | TTPPVLDSDGSFFLYSRLTVDKSRWQEGNVFSCSVMHE | |
| | ALHNHYTQKSLSLSLGK | |
| Human IgG4(S228P) | ASTKGPSVFPLAPCSRSTSESTAALGCLVKDYFPEPVTVS | 11 |
| | WNSGALTSGVHTFPAVLQSSGLYSLSSVVTVPSSSLGTK | |
| | TYTCNVDHKPSNTKVDKRVESKYGPPCPPCPAPEFLGGP | |
| | SVFLFPPKPKDTLMISRTPEVTCVVVDVSQEDPEVQFNW | |
| | YVDGVEVHNAKTKPREEQFNSTYRVVSVLTVLHQDWL | |
| | NGKEYKCKVSNKGLPSSIEKTISKAKGQPREPQVYTLPPS | |
| | QEEMTKNQVSLTCLVKGFYPSDIAVEWESNGQPENNYK | |
| | TTPPVLDSDGSFFLYSRLTVDKSRWQEGNVFSCSVMHE | |
| | ALHNHYTQKSLSLSLGK | |
| Anti-PD-L1 HCDR1 | TYAIS | 12 |
| (Kabat) | | |
| Anti-PD-L1 HCDR2 | GIIPIFGKAHYAQKFQG | 13 |
| (Kabat) | | |
| Anti-PD-L1 HCDR3 | KFHFVSGSPFGMDV | 14 |
| (Kabat) | | |
| Anti-PD-L1 HCDR1 | GDTFSTY | 34 |
| (Chothia) | | |
| Anti-PD-L1 HCDR2 | IPIFGK | 35 |
| (Chothia) | | |
| Anti-PD-L1 HCDR3 | KFHFVSGSPFGMDV | 14 |
| (Chothia) | | |
| Anti-PD-L1 HCDR1 | GDTFSTYA | 36 |
| (IMGT) | | |
| Anti-PD-L1 HCDR2 | IIPIFGKA | 37 |
| (IMGT) | | |
| Anti-PD-L1 HCDR3 | ARKFHFVSGSPFGMDV | 38 |
| (IMGT) | | |
| Anti-PD-L1 LCDR1 | RASQSVSSYLA | 15 |
| (Kabat) | | |
| Anti-PD-L1 LCDR2 | DASNRAT | 16 |
| (Kabat) | | |
| Anti-PD-L1 LCDR3 | QQRSNWPT | 17 |
| (Kabat) | | |
| Anti-PD-L1 LCDR1 | RASQSVSSYLA | 15 |
| (Chothia) | | |
| Anti-PD-L1 LCDR2 | DASNRAT | 16 |
| (Chothia) | | |
| Anti-PD-L1 LCDR3 | QQRSNWPT | 17 |
| (Chothia) | | |
| Anti-PD-L1 LCDR1 | QSVSSY | 39 |
| (IMGT) | | |
| Anti-PD-L1 LCDR2 | DA | |
| (IMGT) | | |
| Anti-PD-L1 LCDR3 | QQRSNWPT | 17 |
| (IMGT) | | |
| Anti-CD89 HCDR1 | SYVLH | 18 |
| (Kabat) | | |
| Anti-CD89 HCDR2 | VISDDGRNKYFADSVKG | 19 |
| (Kabat) | | |
| Anti-CD89 HCDR3 | EGYSGSWFDY | 20 |
| (Kabat) | | |
| Anti-CD89 HCDR1 | GFTFSSY | 40 |
| (Chothia) | | |
| Anti-CD89 HCDR2 | SDDGRN | 41 |
| (Chothia) | | |
| Anti-CD89 HCDR3 | EGYSGSWFDY | 20 |
| (Chothia) | | |
| Anti-CD89 HCDR1 | GFTFSSYV | 42 |
| (IMGT) | | |
| Anti-CD89 HCDR2 | ISDDGRNK | 43 |
| (IMGT) | | |
| Anti-CD89 HCDR3 | VREGYSGSWFDY | 44 |
| (IMGT) | | |
| Anti-CD89 LCDR1 | RASQGISSALA | 21 |
| (Kabat) | | |
| Anti-CD89 LCDR2 | GASSLEG | 22 |
| (Kabat) | | |
| Anti-CD89 LCDR3 | QQFNSYPFT | 23 |
| (Kabat) | | |
| Anti-CD89 LCDR1 | RASQGISSALA | 21 |
| (Chothia) | | |
| Anti-CD89 LCDR2 | GASSLEG | 22 |
| (Chothia) | | |
| Anti-CD89 LCDR3 | QQFNSYPFT | 23 |
| (Chothia) | | |
| Anti-CD89 LCDR1 | QGISSA | 45 |
| (IMGT) | | |
| Anti-CD89 LCDR2 | GA | |
| (IMGT) | | |
| Anti-CD89 LCDR3 | QQFNSYPFT | 23 |
| (IMGT) | | |
| Anti-PD-L1 VH | QVQLVQSGAEVKKPGSSVKVSCKTSGDTFSTYAISWVR | 24 |
| domain | QAPGQGLEWMGGIIPIFGKAHYAQKFQGRVTITADESTS | |
| | TAYMELSSLRSEDTAVYFCARKFHFVSGSPFGMDVWGQ | |
| | GTTVTVSS | |
| Anti-PD-L1 VL | EIVLTQSPATLSLSPGERATLSCRASQSVSSYLAWYQQK | 25 |
| domain | PGQAPRLLIYDASNRATGIPARFSGSGSGTDFTLTISSLEP | |
| | EDFAVYYCQQRSNWPTFGQGTKVEIK | |
| Anti-CD89 VH | QVQLVESGGGVVQPGRSLRLSCAASGFTFSSYVLHWVR | 26 |
| domain | QAPGKGLDWVAVISDDGRNKYFADSVKGRFTISRDNSK | |
| | NTLYLQMNSLRAEDTAVYYCVREGYSGSWFDYWGQG | |
| | TLVTVSS | |
| Anti-CD89 VL | AIQLTQSPSSLSASVGDRVTITCRASQGISSALAWYQQKP | 27 |
| domain | GKAPKLLIYGASSLEGGVPSRFSGSGSGTDFTLTISSLQPE | |
| | DFATYYCQQFNSYPFTFGPGTKVDIK | |

In the VH and VL domain sequences, the CDR sequences are underlined.

TABLE 2
Full-length sequences of illustrative heavy and light chains

| Name | SEQ ID NO | Amino Acid sequence |
|---|---|---|
| 1L | 28 | EIVLTQSPATLSLSPGERATLSCRASQSVSSYLAWYQQKPGQAPRLLIY |
| | | DASNRATGIPARFSGSGSGTDFTLTISSLEPEDFAVYYCQQRSNWPTFG |
| | | QGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYA |
| | | CEVTHQGLSSPVTKSFNRGECGPAPLGLGGGSAIQLTQSPSSLSASVG |
| | | DRVTITCRASQGISSALAWYQQKPGKAPKLLIYGASSLEGGVPSRFSG |
| | | SGSGTDFTLTISSLQPEDFATYYCQQFNSYPFTFGPGTKVDIKRTVAAP |
| | | SVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKS |
| | | FNRGEC |
| 1LX | 29 | EIVLTQSPATLSLSPGERATLSCRASQSVSSYLAWYQQKPGQAPRLLIY |
| | | DASNRATGIPARFSGSGSGTDFTLTISSLEPEDFAVYYCQQRSNWPTFG |
| | | QGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYA |
| | | CEVTHQGLSSPVTKSFNRGECGPAPLGLGGGGSAIQLTQSPSSLSASV |
| | | GDRVTITCRASQGISSALAWYQQKPGKAPKLLIYGASSLEGGVPSRFS |
| | | GSGSGTDFTLTISSLQPEDFATYYCQQFNSYPFTFGPGTKVDIKRTVAA |
| | | PSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVT |
| | | KSFNRGEC |
| 1H | 30 | QVQLVQSGAEVKKPGSSVKVSCKTSGDTFSTYAISWVRQAPGQGLE |
| | | WMGGIIPIFGKAHYAQKFQGRVTITADESTSTAYMELSSLRSEDTAVY |
| | | FCARKFHFVSGSPFGMDVWGQGTTVTVSSASTKGPSVFPLAPSSKSTS |
| | | GGTAALGCLVKDYFPEPVTVSWNSGALTSGVHTFPAVLQSSGLYSLS |
| | | SVVTVPSSSLGTQTYICNVNHKPSNTKVDKKVEPKSCGPAPELLGGGS |
| | | QVQLVESGGGVVQPGRSLRLSCAASGFTFSSYVLHWVRQAPGKGLD |
| | | WVAVISDDGRNKYFADSVKGRFTISRDNSKNTLYLQMNSLRAEDTA |
| | | VYYCVREGYSGSWFDYWGQGTLVTVSSASTKGPSVFPLAPSSKSTSG |
| | | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAP |
| | | EAAGAPSVFLFPPKPKDTLMISRTPEVTCVVVDVSHEDPEVKFNWYV |
| | | DGVEVHNAKTKPREEQYNSTYRVVSVLTVLHQDWLNGKEYKCKVS |
| | | NKALPAPIEKTISKAKGQPREPQVYTLPPCRDELTKNQVSLWCLVKGF |
| | | YPSDIAVEWESNGQPENNYKTTPPVLDSDGSFFLYSKLTVDKSRWQQ |
| | | GNVFSCSVMHEALHNHYTQKSLSLSPGK |
| 1HX | 31 | QVQLVQSGAEVKKPGSSVKVSCKTSGDTFSTYAISWVRQAPGQGLE |
| | | WMGGIIPIFGKAHYAQKFQGRVTITADESTSTAYMELSSLRSEDTAVY |
| | | FCARKFHFVSGSPFGMDVWGQGTTVTVSSASTKGPSVFPLAPSSKSTS |
| | | GGTAALGCLVKDYFPEPVTVSWNSGALTSGVHTFPAVLQSSGLYSLS |
| | | SVVTVPSSSLGTQTYICNVNHKPSNTKVDKKVEPKSCGPAPELLGGG |
| | | GSQVQLVESGGGVVQPGRSLRLSCAASGFTFSSYVLHWVRQAPGKG |
| | | LDWVAVISDDGRNKYFADSVKGRFTISRDNSKNTLYLQMNSLRAEDT |
| | | AVYYCVREGYSGSWFDYWGQGTLVTVSSASTKGPSVFPLAPSSKSTS |
| | | GGTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPCPA |
| | | PEAAGAPSVFLFPPKPKDTLMISRTPEVTCVVVDVSHEDPEVKFNWY |
| | | VDGVEVHNAKTKPREEQYNSTYRVVSVLTVLHQDWLNGKEYKCKV |
| | | SNKALPAPIEKTISKAKGQPREPQVYTLPPCRDELTKNQVSLWCLVKG |
| | | FYPSDIAVEWESNGQPENNYKTTPPVLDSDGSFFLYSKLTVDKSRWQ |
| | | QGNVFSCSVMHEALHNHYTQKSLSLSPGK |
| Fc- | 32 | SCPPCPAPEAAGAPSVFLFPPKPKDTLMISRTPEVTCVVVDVSHEDPEV |
| stump | | KFNWYVDGVEVHNAKTKPREEQYNSTYRVVSVLTVLHQDWLNGKE |
| | | YKCKVSNKALPAPIEKTISKAKGQPREPQVCTLPPSRDELTKNQVSLS |
| | | CAVKGFYPSDIAVEWESNGQPENNYKTTPPVLDSDGSFFLVSKLTVD |
| | | KSRWQQGNVFSCSVMHEALHNHYTQKSLSLSPGK |

The anti-PD-L1/anti-CD89 protein design may be based on sequences derived from IgG1, IgG2, IgG3, IgG4, IgE, IgM, or IgA and may or may not have effector function capacity.

Proteins disclosed herein comprise domains and regions of antibody molecules. The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable domain (abbreviated herein as VH domain) and a heavy chain constant region. The heavy chain constant region comprises three domains, CH1, CH2 and CH3. IgG, IgA, and IgD constant regions comprise a flexible hinge region between the CH1 domain and the CH2 domain. Each light chain comprises a light chain variable domain (abbreviated herein as VL domain) and a light chain constant region. The light chain constant region comprises one domain, CL. The VH and VL domains can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4. The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, Chothia, C. et al. (1987) J. Mol. Biol. 196:901-917, or IMGT Lefranc MP. Unique database numbering system for immunogenetic analysis. Immunol Today. 1997 November; 18(11):509.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU index as in Kabat. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins.

A protein provided herein comprises two Fab fragments. A Fab fragment is a monovalent antigen-binding fragment consisting of the VL, VH, CL and CH1 domains.

Immunoglobulin molecules can be of any type (e.g., IgG, IgE, IgM, IgD, IgA or IgY) and class (e.g., IgG1, IgG2, IgG3, IgG4, IgA1 or IgA2) or subclass. IgG, IgD, and IgE antibodies generally contain two identical heavy chains and two identical light chains and two antigen combining domains, each composed of a VH) and a VL. Generally, IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

As used herein, the terms “immunological binding” and “immunological binding properties” refer to the non-covalent interactions of the type which occur between an immunoglobulin molecule (e.g., antibody or antigen-binding portion thereof), or a protein comprising an immunoglobulin-derived binding domain(s) and an antigen for which the immunoglobulin or protein is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity and is equal to the dissociation constant K_{d}. (See, Davies et al. (1990) Annual Rev Biochem 59:439-473). An antibody or antigen-binding portion provided herein is said to specifically bind PD-L1 or CD89 when the equilibrium binding constant (K_{d}) is ≤10 μM, preferably ≤10 nM, more preferably ≤10 nM, and most preferably ≤100 pM to about 1 pM, as measured by assays such as radioligand binding assays or similar assays known to those skilled in the art. One method for determining the K_{d} of an antibody is by using surface plasmon resonance (SPR), typically using a biosensor system such as a Biacore® system.

Functionally, the binding affinity of a protein provided herein may be within the range of 10⁻⁵M to 10⁻¹² M. For example, the binding affinity of a protein provided herein is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10-8 M to 10⁻¹¹ M, from 10⁻⁹ M to 10⁻¹¹ M, from 10⁻¹⁰ M to 10⁻¹¹ M, from 10⁻⁵ M to 10⁻¹⁰ M, from 10⁻⁶ M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M or from 10⁻⁵ M to 10⁻⁶ M.

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD89 VH domain, and a second CH1 domain; wherein the light chain comprises, in N-terminus to C-terminus order, an anti-PD-L1 light chain variable (VL) domain, a first immunoglobulin light chain constant region, a second linker, an anti-CD89 VL domain, and a second immunoglobulin light chain constant region.

In some embodiments, the heavy chain comprises in N-terminus to C-terminus order, the anti-PD-L1 VH domain, the first CH1 domain, the first linker, the anti-CD89 VH domain, the second CH1 domain, a hinge, a CH2 domain, and a CH3 domain.

In some embodiments, the protein further comprises a third polypeptide chain comprising a hinge and a Fc region. In some embodiments, the third polypeptide chain comprises the amino acid sequence of SEQ ID NO: 32.

In some embodiments, the first linker comprises the amino acid sequence of SEQ ID NO: 1, SEQ ID NO: 2, SEQ ID NO: 3, or SEQ ID NO: 4. In some embodiments, the second linker comprises the amino acid sequence of SEQ ID NO: 1, SEQ ID NO: 2, SEQ ID NO: 3, or SEQ ID NO: 4.

In some embodiments, the anti-PD-L1 VH domain comprises a HCDR1 comprising the amino acid sequence of SEQ ID NO: 12, a HCDR2 comprising the amino acid sequence of SEQ ID NO: 13, and a HCDR3 comprising the amino acid sequence of SEQ ID NO: 14; the anti-PD-L1 VL domain comprises a LCDR1 comprising the amino acid sequence of SEQ ID NO: 15, a LCDR2 comprising the amino acid sequence of SEQ ID NO: 16, and a LCDR3 comprising the amino acid sequence of SEQ ID NO: 17; the anti-CD89 VH domain comprises a HCDR1 comprising the amino acid sequence of SEQ ID NO: 18, a HCDR2 comprising the amino acid sequence of SEQ ID NO: 19, and a HCDR3 comprising the amino acid sequence of SEQ ID NO: 20; and the anti-CD89 VL domain comprises a LCDR1 comprising the amino acid sequence of SEQ ID NO: 21, a LCDR2 comprising the amino acid sequence of SEQ ID NO: 22, and a LCDR3 comprising the amino acid sequence of SEQ ID NO: 23.

In some embodiments, the anti-PD-L1 VH domain comprises the amino acid sequence of SEQ ID NO: 24, and the anti-PD-L1 VL domain comprises the amino acid sequence of SEQ ID NO: 25.

In some embodiments, the anti-CD89 VH domain comprises the amino acid sequence of SEQ ID NO: 26.

In some embodiments, the anti-CD89 VL domain comprises the amino acid sequence of SEQ ID NO: 27.

In some embodiments, the heavy chain comprises the amino acid sequence of SEQ ID NO: 30 or SEQ ID NO: 31. In some embodiments, the heavy chain comprises an amino acid sequence that is at least 90%, 91%, 92%, 93%, 94%, 95%, 96%, 97%, 98% or 99% identical to the amino acid sequence of SEQ ID NO: 30 or SEQ ID NO: 31.

In some embodiments, the light chain comprises the amino acid sequence of SEQ ID NO: 28 or SEQ ID NO: 29. In some embodiments, the heavy chain comprises an amino acid sequence that is at least 90%, 91%, 92%, 93%, 94%, 95%, 96%, 97%, 98% or 99% identical to the amino acid sequence of SEQ ID NO: 28 or SEQ ID NO: 29.

In some embodiments, provided herein is a protein wherein
(a) the heavy chain comprises the amino acid sequence of SEQ ID NO: 30, the light chain comprises the amino acid sequence of SEQ ID NO: 28, and the third polypeptide chain comprises the amino acid sequence of SEQ ID NO: 32; or
(b) the heavy chain comprises the amino acid sequence of SEQ ID NO: 31, the light chain comprises the amino acid sequence of SEQ ID NO: 29, and the third polypeptide chain comprises the amino acid sequence of SEQ ID NO: 32.

Also provided herein is a protein comprising a heavy chain and a light chain, wherein the heavy chain comprises an amino acid sequence provided herein, with 1, 2 or 3 conservative amino acid substitutions; and wherein the light chain comprises an amino acid sequence provided herein, with 1, 2 or 3 conservative amino acid substitutions. In some embodiments, conservative amino acid substitutions are made only in the FR sequences and not in the CDR sequences. In some embodiments, conservative amino acid substitutions are not made in the first linker or the second linker sequences.

In some embodiments, a protein provided herein comprises an immunoglobulin heavy chain constant region at the C-terminus of the heavy chain. In some embodiments, the immunoglobulin heavy chain constant region is IgG, IgE, IgM, IgD, IgA or IgY. In some embodiments, the immunoglobulin heavy chain constant region is IgG1, IgG2, IgG3, IgG4, IgA1 or IgA2. In some embodiments, the immunoglobulin heavy chain constant region is IgG1. In some embodiments, the immunoglobulin heavy chain constant region is immunologically inert. In some embodiments, the immunoglobulin heavy chain constant region comprises one or more mutations to reduce or prevent FcγR binding, antibody-dependent cell-mediated cytotoxicity (ADCC) activity, antibody-dependent cellular phagocytosis (ADCP), and/or complement-dependent cytotoxicity (CDC) activity. In some embodiments, the immunoglobulin heavy chain constant region is a wild-type human IgG1 constant region, a wild-type human IgG2 constant region, a wild-type human IgG4 constant region, a human IgG1 constant region comprising the amino acid substitutions L234A, L235A and G237A, a human IgG1 constant region comprising the amino acid substitutions L234A, L235A, G237A and P331S or a human IgG4 constant region comprising the amino acid substitution S228P, wherein numbering is according to the EU index as in Kabat. In some embodiments, a position of an amino acid residue in a constant region of an immunoglobulin molecule is numbered according to the EU index as in Kabat (Ward et al., 1995 Therap. Immunol. 2:77-94).

In some embodiments, a protein provided herein may comprise an immunoglobulin light chain constant region that is a kappa light chain. In some embodiments a kappa light chain comprises SEQ ID NO: 7.

In some embodiments, a protein provided herein may comprise an immunoglobulin light chain constant region that is a lambda light chain.

In some embodiments, a protein provided herein may comprise an immunoglobulin heavy chain constant region comprising an amino acid sequence of an Fc region of human IgG4, human IgG4(S228P), human IgG2, human IgG1, human IgG1 effector null. For example, the human IgG4(S228P) Fc region comprises the following substitution compared to the wild-type human IgG4 Fc region: S228P. For example, the human IgG1 effector null Fc region comprises the following substitutions compared to the wild-type human IgG1 Fc region: L234A, L235A and G237A. In some embodiments, a protein may comprise an immunoglobulin heavy chain constant region comprising the amino acid sequence of any one of SEQ ID NO: 5, SEQ ID NO: 6, SEQ ID NO: 8, SEQ ID NO: 9, SEQ ID NO: 10 and SEQ ID NO: 11.

Provided herein is an immunoconjugate comprising a protein disclosed herein linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Examples of suitable therapeutic agents include, but are not limited to, immunomodulatory agents, cytotoxins, radioisotopes, chemotherapeutic agents, anti-angiogenic agents, antiproliferative agents, pro-apoptotic agents, and cytostatic and cytolytic enzymes (for example, RNAses). Further therapeutic agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above terms.

Examples of suitable therapeutic agents for use in immunoconjugates include, but are not limited to, JAK kinase inhibitors, taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., ADEPT), radioisotopes, photosensitizers may also be used in immunoconjugates. In addition, conjugates can be made using secondary carriers as the cytotoxic agent, such as liposomes or polymers, Suitable cytotoxins include an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins.

Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluhdine, pentostatin, broxuhdine, capecitabine, cladhbine, decitabine, floxuhdine, fludarabine, gougerotin, puromycin, tegafur, tiazofuhn, adhamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

Suitable immunomodulatory agents include anti-hormones that block hormone action on tumors and immunosuppressive agents that suppress cytokine production, down-regulate self-antigen expression, or mask MHC antigens.

TABLE 3
Chain sequence combinations used in illustrative proteins

| Name | Heavy Chain Construct | Light Chain Construct | Heavy Chain Construct 2 |
|---|---|---|---|
| LPD89-1 | 1H (SEQ ID NO: 30) | 1L (SEQ ID NO: 28) | Fc-stump (SEQ ID NO: 32) |
| LPD89-2 | 1HX (SEQ ID NO: 31) | 1LX (SEQ ID NO: 29 | Fc-stump (SEQ ID NO: 32) |

### Pharmaceutical Compositions

The activatable proteins provided herein (also referred to herein as “active compounds”) can be incorporated into pharmaceutical compositions suitable for administration. Such compositions typically comprise a protein (or an immunoconjugate comprising said protein), and a pharmaceutically acceptable carrier, diluent or excipient. Such materials should be non-toxic and should not interfere with the efficacy of the protein. The precise nature of the carrier or other material will depend on the route of administration, which may be by injection, bolus, infusion, or any other suitable route, as discussed below.

As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions. A pharmaceutically acceptable carrier, diluent or excipient may be a compound or a combination of compounds that does not provoke secondary reactions and that allows, for example, facilitation of the administration of the protein, an increase in its lifespan and/or in its efficacy in the body or an increase in its solubility in solution.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, NJ) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the protein may be provided in a lyophilized form for reconstitution prior to administration. For example, lyophilized antibody molecules may be reconstituted in sterile water and mixed with saline prior to administration to an individual.

The pharmaceutical compositions provided herein can be included in a container, pack, or dispenser together with instructions for administration.

### Nucleic Acid Molecules, Vectors, Host Cells and Methods of Producing Proteins

Provided herein is a nucleic acid molecule (e.g., an isolated nucleic acid molecule) encoding an amino acid sequence of a protein disclosed herein (or an amino acid sequence of a (i) VH domain, (ii) a VL domain, or (iii) both a VH domain and a VL domain of a protein). Further provided herein is a nucleic acid molecule (e.g., an isolated nucleic acid molecule) encoding (i) a heavy chain, (ii) a light chain, or (iii) both a heavy chain and a light chain of a protein disclosed herein. In some embodiments, a nucleic acid molecule encoding a VH domain, a VL domain, a heavy chain or a light chain comprises a signal sequence (or encodes a leader peptide). In some embodiments, a nucleic acid molecule encoding a VH domain, a VL domain, a heavy chain or a light chain does not comprise a signal sequence (or does not encode a leader peptide).

Also provided herein is an expression vector comprising a nucleic acid molecule described herein. In certain vectors, a nucleic acid molecule is operatively linked to one or more regulatory sequences suitable for expression of the nucleic acid segment in a host cell. In some cases, an expression vector comprises sequences that mediate replication and comprises one or more selectable markers. As used herein, “vector” means a construct that is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Provided herein is a recombinant host cell comprising an expression vector or a nucleic acid molecule disclosed herein. A “host cell” includes an individual cell, a cell line or cell culture that can be or has been a recipient for vector(s) for incorporation of polynucleotide inserts. Host cells include progeny of a single host cell. The progeny may not necessarily be completely identical (in morphology or in genomic DNA complement) to the original parent cell due to natural, accidental, or deliberate mutation. An expression vector can be transfected into a host cell by standard techniques. Non-limiting examples include electroporation, calcium-phosphate precipitation, DEAE-dextran transfection and the like. In some embodiments, a recombinant host cell comprises a single vector or a single nucleic acid molecule encoding both a heavy chain and a light chain of a protein disclosed herein. In some embodiments, a recombinant host cell comprises (i) a first vector or a first nucleic acid molecule encoding a heavy chain of a protein disclosed herein and (ii) a second vector or a second nucleic acid molecule encoding a light chain of a protein disclosed herein.

Protein molecules of the invention, or portions thereof, can be produced using techniques well known in the art, for example, recombinant technologies, phage display technologies, synthetic technologies, computational technologies or combinations of such technologies or other technologies readily known in the art.

Further provided herein is a method for producing a protein disclosed herein, the method comprising: culturing a recombinant host cell comprising an expression vector described herein under conditions whereby the nucleic acid segment is expressed, thereby producing the protein. The protein may then be isolated from the host cell or culture. Provided herein is a method of producing a protein, the method comprising: culturing a recombinant host cell comprising an expression vector disclosed herein under conditions whereby the nucleic acid molecule is expressed, thereby producing the protein; and isolating the protein from the host cell or culture.

Proteins disclosed herein can be produced by any of a variety of methods known to those skilled in the art. In certain embodiments, proteins disclosed herein can be produced recombinantly. For example, nucleic acid sequences encoding one or more of the heavy chains or light chains provided herein, or portions thereof, may be introduced into a bacterial cell (e.g., E. coli, B. subtilis) or a eukaryotic cell (e.g., a yeast such as S. cerevisiae, or a mammalian cell such as a CHO cell line, various Cos cell lines, a HeLa cell, a HEK293 cell, various myeloma cell lines, or a transformed B-cell or hybridoma), or into an in vitro translation system, and the translated polypeptide may be isolated. In some embodiments, light chain proteins and heavy chain proteins are produced in a cell with a signal sequence that is removed upon production of a mature protein disclosed herein.

Those skilled in the art will be able to determine whether a protein comprising a given polypeptide sequence binds to PD-L1 protein and/or CD89 protein using standard methodologies, for example, Western blots, ELISA, and the like.

### Medical Uses of Activatable Proteins

Provided herein are methods and uses of activatable proteins, immunoconjugates, and pharmaceutical compositions disclosed herein for providing a therapeutic benefit to a subject with cancer.

An activatable protein, immunoconjugate, or pharmaceutical composition disclosed herein may be used in a method of treatment of the human or animal body, including prophylactic or preventative treatment (e.g., treatment before the onset of a condition in a subject to reduce the risk of the condition occurring in the subject; delay its onset; or reduce its severity after onset). The method of treatment may comprise administering the protein, immunoconjugate, or pharmaceutical composition to a subject in need thereof.

Provided herein is a method for enhancing an anti-cancer immune response in a subject, the method comprising administering to the subject a therapeutically effective amount of a protein, an immunoconjugate, or a pharmaceutical composition disclosed herein. In some embodiments, an anti-cancer immune response is a T cell response. In some embodiments, an anti-cancer immune response is a complement response.

Provided herein is a method for treating cancer in a subject, the method comprising administering to the subject a therapeutically effective amount of a protein, an immunoconjugate, or a pharmaceutical composition disclosed herein.

Provided herein is a method for ameliorating a symptom of cancer in a subject, the method comprising administering to the subject a therapeutically effective amount of a protein, immunoconjugate, or a pharmaceutical composition disclosed herein.

Provided herein is a method for reducing the size of a tumor in a subject, the method comprising administering to the subject a therapeutically effective amount of a protein, an immunoconjugate, or a pharmaceutical composition disclosed herein.

Provided herein is a method for inhibiting the growth of a tumor in a subject, the method comprising administering to the subject a therapeutically effective amount of a protein, an immunoconjugate, or a pharmaceutical composition disclosed herein.

In some embodiments, the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

In some embodiments, the cancer is a solid tumor. In some embodiments, the cancer is a hematological cancer.

In some embodiments, a cancer of hematological tissues is a lymphoma. In some embodiments, the cancer is mantle cell lymphoma, acute lymphoblastic leukemia, chronic lymphocytic leukemia, Non-Hodgkin's lymphoma, Hodgkin's lymphoma, acute myeloid leukemia (AML), B-lymphoid leukemia, blastic plasmocytoid dendritic neoplasm (BPDCN), or hairy cell leukemia.

As used herein, the term “effective amount” or “therapeutically effective amount” refers to the amount of a pharmaceutical agent, e.g., a protein, an immunoconjugate, or a pharmaceutical composition disclosed herein, which is sufficient to reduce or ameliorate the severity and/or duration of a cancer, or one or more symptoms thereof, prevent the advancement of a disease, cause regression of a disease, prevent the recurrence, development, onset or progression of one or more symptoms associated with a disease, or enhance or improve the prophylactic or therapeutic effect(s) of another related therapy (e.g., prophylactic or therapeutic agent) for a cancer.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g., decisions on dosage etc., is within the responsibility of general practitioners and other medical doctors and may depend on the severity of the symptoms and/or progression of a disease being treated. Appropriate doses of antibody-based protein molecules are well known in the art (Ledermann J. A. et al., 1991, Int. J. Cancer 47: 659-664; Bagshawe K. D. et al., 1991, Antibody, Immunoconjugates and Radiopharmaceuticals 4: 915-922). Specific dosages may be indicated herein or in the Physician's Desk Reference (2003) as appropriate for the type of medicament being administered may be used. A therapeutically effective amount or suitable dose of an antibody-based protein molecule may be determined by comparing its in vitro activity and in vivo activity in an animal model. Methods for extrapolation of effective dosages in mice and other test animals to humans are known. The precise dose will depend upon a number of factors, including whether the antibody-based protein is for prevention or for treatment, the size and location of the area to be treated, the precise nature of the antibody-based protein, and the nature of any detectable label or other molecule attached to the antibody-based protein.

A typical protein dose will be in the range 100 μg to 1 g for systemic applications, and 1 μg to 1 mg for intradermal injection. An initial higher loading dose, followed by one or more lower doses, may be administered. In some embodiments, the protein is an IgG1 or IgG4 isotype. A dose for a single treatment of an adult subject may be proportionally adjusted for children and infants. Treatments may be repeated at daily, twice-weekly, weekly or monthly intervals, at the discretion of the physician. The treatment schedule for a subject may be dependent on the pharmacokinetic and pharmacodynamic properties of the protein composition, the route of administration and the nature of the condition being treated.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above.

In some embodiments, a protein, an immunoconjugate, or a pharmaceutical composition disclosed herein may be administered as a sub-cutaneous injection. Sub-cutaneous injections may be administered using an auto-injector, for example for long term prophylaxis/treatment.

In some embodiments, the therapeutic effect of a protein, an immunoconjugate, or a pharmaceutical composition disclosed herein may persist for several half-lives, depending on the dose. For example, the therapeutic effect of a single dose of a protein, an immunoconjugate, or a pharmaceutical composition disclosed herein may persist in a subject for 1 month or more, 2 months or more, 3 months or more, 4 months or more, 5 months or more, or 6 months or more.

In some embodiments, a subject may be treated with a protein, an immunoconjugate, or a pharmaceutical composition disclosed herein and an additional therapeutic agent or therapy that is used to treat a cancer or a symptom or complication of a cancer. The protein, immunoconjugate, or pharmaceutical composition disclosed herein and the additional therapeutic agent or therapy may be administered simultaneously or sequentially.

In some embodiments, a subject is a human, a non-human primate, a pig, a horse, a cow, a dog, a cat, a guinea pig, a mouse or a rat. In some embodiments, a subject is an adult human. In some embodiments, a subject is a pediatric human.

Further provided herein is a protein, an immunoconjugate, or a pharmaceutical composition disclosed herein, for use in the treatment of a disease or a disorder.

Provided herein is a protein, an immunoconjugate, or a pharmaceutical composition disclosed herein, for use as a medicament.

### Definitions

Unless otherwise noted, the terms used herein have definitions as ordinarily used in the art. Some terms are defined below, and additional definitions can be found within the rest of the detailed description.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “sequence identity” refers to the extent to which two optimally aligned polynucleotides or polypeptide sequences are invariant throughout a window of alignment of residues, e.g., nucleotides or amino acids. An “identity fraction” for aligned segments of a test sequence and a reference sequence is the number of identical residues which are shared by the two aligned sequences divided by the total number of residues in the reference sequence segment, i.e., the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 Oct. 11) Molecular Systems Biology 7:539. For the purposes of calculating identity to the sequence, extensions, such as tags, are not included.

As used herein, the term “HCDR” refers to a heavy chain complementarity determining region. As used herein, the term “LCDR” refers to a light chain complementarity determining region.

The terms “amino-terminal”, “N-terminus”, “carboxyl-terminal”, and “C-terminus” are used herein to denote positions within polypeptide chains. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl-terminus of the reference sequence but is not necessarily at the carboxyl-terminus of the complete polypeptide.

As used herein, the term “conservative substitution” refers to replacement of an amino acid with another amino acid which does not significantly deleteriously change the functional activity. A preferred example of a “conservative substitution” is the replacement of one amino acid with another amino acid which has a value ≥0 in the following BLOSUM 62 substitution matrix (see Henikoff & Henikoff, 1992, PNAS 89: 10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4. |

The term “immunoconjugate” refer to a protein of the disclosure that is conjugated to a cytotoxic, a cytostatic and/or a therapeutic agent.

The term “isolated molecule” (where the molecule is, for example, a protein, a nucleic acid, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The terms “inhibit”, “block”, or “neutralize”, as used herein with respect to bioactivity of a protein disclosed herein means the ability of the protein to substantially antagonize, prohibit, prevent, restrain, slow, disrupt, eliminate, stop, reduce or reverse for example progression, strength, or severity of that which is being inhibited including, but not limited to, the binding of PD-L1 to PD-1, or the binding of CD89 to IgA.

As used herein, the terms “treat,” “treating” or “treatment of” (and grammatical variations thereof) mean that the severity of the subject's condition is reduced, at least partially improved or stabilized and/or that some alleviation, mitigation, decrease or stabilization in at least one clinical symptom is achieved and/or there is a delay in the progression of the disease or disorder.

PD-L1 is also known as programmed cell death ligand 1, CD274, B7-H, B7H1, PDCD1L1, PDCD1LG1, PDL1, and hPD-L1.

CD89 is also known as cluster of differentiation 89 and TP44. CD89 is a transmembrane glycoprotein that binds both subclasses of IgA.

As used herein, the terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the compositions and/or methods described herein. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the compositions and/or methods described herein.

As used herein, a “therapeutically effective amount” is the amount of a protein or a pharmaceutical composition provided herein that is effective to treat or prevent a disease or disorder in a subject or to ameliorate a sign or symptom thereof. The “therapeutically effective amount” may vary depending, for example, on the disease and/or symptoms of the disease, severity of the disease and/or symptoms of the disease or disorder, the age, weight, and/or health of the patient to be treated, and the judgment of the prescribing physician.

As used herein and unless otherwise stated, the terms “hinge linker”, “linker”, “hinge”, “first linker”, “second linker”, “lower hinge linker” (“LHL”), “inter-Fab linker”, and derivations thereof, in plural or singular form, refer to a sequence, for example derived from an immunoglobulin hinge region, that can link two polypeptides, for example polypeptides of different Fab regions, and is separate from any hinge sequence in an immunoglobulin hinge region that may be part of a protein of the present invention.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

The section headings used herein are for organizational purposes only and are not to be construed as limiting the subject matter described.

The disclosure will be further clarified by the following example, which is intended to be purely exemplary of the disclosure and in no way limiting.

### EXAMPLE

### Generation of Optimized Conditionally Active Therapeutic Proteins

### Introduction

In this example, we successfully generated optimized conditionally active proteins. These conditionally active proteins were well expressed.

### Materials and Methods

### Cloning, Transient Expression, Purification and Characterization of Proteins

Polypeptide-encoding DNA sequences were cloned via restriction-ligation cloning into separate human IgG1 heavy and light-chain constant region-encoding expression cassettes in separate plasmid vectors, to create activatable constructs for expression. Proteins were expressed in CHO cells and purified from culture supernatants via a combination of Protein A affinity chromatography (ProA), Ion Exchange Chromatography (IEX) and/or Size Exclusion Chromatography (SEC). Purified proteins were characterized by SEC, SDS-PAGE and mass spectrometry.

### Results and Discussion

### Protein Construct Design Cloning, Expression, and Characterization

To produce proteins for functional testing, DNA cassettes for each construct type were designed using combinations of the variable domain, constant domain and linker sequences found in Table 1. These humanized variants were then combined with PD-L1 v domains, constant domains and linkers to form full-length heavy and light chain sequences (Table 2). Using these full-length chains, 2 initial designs were synthesized and cloned into expression vectors encoding human IgG1 heavy and light chain sequences and a free hinge-Fc fragment (Fc Stump, Table 2). The anti-PD-L1 variable domain sequences used in the protein construct disclosed herein are the variable domain sequences provided in U.S. Pat. No. 7,943,743 B2. The anti-CD89 variable domain sequences used in the protein construct disclosed herein are the variable domain sequences provided in U.S. 2003/0082643 A1. The proteins were produced by transient transfection of CHO cells, then purified by proA, IEX and/or SEC. After the ProA step, proteins were examined for yield and uniformity by SEC (Table 4). Fully purified proteins demonstrated high purity (>95%) and uniformity by analytical SEC, demonstrating that the best-behaved constructs can be expressed and purified as an intact, stable product, in a single process. Table 4: Productivity analyses for selected proteins

| | Main | | |
|---|---|---|---|
| Name | Product (%) | HMW (%) | LMW (%) |
| LPD89-1 | 66 | 11 | 23 |
| LPD89-2 | 70 | 12 | 18 |

HMW = Higher Molecular Weight Product
LMW = Lower Molecular Weight Product

### EMBODIMENTS

1. A protein comprising a first polypeptide chain comprising a heavy chain and a second polypeptide chain comprising a light chain,
   wherein the heavy chain comprises, in N-terminus to C-terminus order, an anti-PD-L 1 heavy chain variable (VH) domain, a first CH1 domain, a first linker, an anti-CD89 VH domain, and a second CH1 domain;
   wherein the light chain comprises, in N-terminus to C-terminus order, an anti-PD-L1 light chain variable (VL) domain, a first immunoglobulin light chain constant region, a second linker, an anti-CD89 VL domain, and a second immunoglobulin light chain constant region.
2. The protein of embodiment 1, wherein the heavy chain comprises in N-terminus to C-terminus order, the anti-PD-L1 VH domain, the first CH1 domain, the first linker, the anti-CD89 VH domain, the second CH1 domain, a hinge, a CH2 domain, and a CH3 domain.
3. The protein of embodiment 1 or 2, wherein the protein further comprises a third polypeptide chain comprising a hinge and a Fc region.
4. The protein of embodiment 3, wherein the third polypeptide chain comprises the amino acid sequence of SEQ ID NO: 32.
5. The protein of any one of embodiments 1-4, wherein the first linker comprises the amino acid sequence of SEQ ID NO: 1, SEQ ID NO: 2, SEQ ID NO: 3, or SEQ ID NO: 4.
6. The protein of any one of embodiments 1-6, wherein the second linker comprises the amino acid sequence of SEQ ID NO: 1, SEQ ID NO: 2, SEQ ID NO: 3, or SEQ ID NO: 4.
7. The protein of any one of embodiments 1-6,
wherein the anti-PD-L1 VH domain comprises a HCDR1 comprising the amino acid sequence of SEQ ID NO: 12, a HCDR2 comprising the amino acid sequence of SEQ ID NO: 13, and a HCDR3 comprising the amino acid sequence of SEQ ID NO: 14;
wherein the anti-PD-L1 VL domain comprises a LCDR1 comprising the amino acid sequence of SEQ ID NO: 15, a LCDR2 comprising the amino acid sequence of SEQ ID NO: 16, and a LCDR3 comprising the amino acid sequence of SEQ ID NO: 17;
wherein the anti-CD89 VH domain comprises a HCDR1 comprising the amino acid sequence of SEQ ID NO: 18, a HCDR2 comprising the amino acid sequence of SEQ ID NO: 19, and a HCDR3 comprising the amino acid sequence of SEQ ID NO: 20;
wherein the anti-CD89 VL domain comprises a LCDR1 comprising the amino acid sequence of SEQ ID NO: 21, a LCDR2 comprising the amino acid sequence of SEQ ID NO: 22, and a LCDR3 comprising the amino acid sequence of SEQ ID NO: 23.
8. The protein of any one of embodiments 1-7, wherein the anti-PD-L1 VH domain comprises the amino acid sequence of SEQ ID NO: 24, and the anti-PD-L1 VL domain comprises the amino acid sequence of SEQ ID NO: 25.
9. The protein of any one of embodiments 1-8, wherein the anti-CD89 VH domain comprises the amino acid sequence of SEQ ID NO: 26.
10. The protein of any one of embodiments 1-9, wherein the anti-CD89 VL domain comprises the amino acid sequence of SEQ ID NO: 27.
11. The protein of any one of embodiments 1-10, wherein the heavy chain comprises the amino acid sequence of SEQ ID NO: 30 or SEQ ID NO: 31.
12. The protein of any one of embodiments 1-11, wherein the light chain comprises the amino acid sequence of SEQ ID NO: 28 or SEQ ID NO: 29.
13. The protein of embodiment 3, wherein
   (a) the heavy chain comprises the amino acid sequence of SEQ ID NO: 30, the light chain comprises the amino acid sequence of SEQ ID NO: 28, and the third polypeptide chain comprises the amino acid sequence of SEQ ID NO: 32; or
   (b) the heavy chain comprises the amino acid sequence of SEQ ID NO: 31, the light chain comprises the amino acid sequence of SEQ ID NO: 29, and the third polypeptide chain comprises the amino acid sequence of SEQ ID NO: 32.
14. The protein of embodiment 2 or 3, wherein the heavy chain comprises an IgG, IgE, IgM, IgD, IgA, or IgY constant region.
15. The protein of embodiment 2 or 3, wherein the heavy chain comprises an IgG1, IgG2, IgG3, IgG4, IgA1 or IgA2 constant region.
16. The protein of embodiment 2 or 3, wherein the heavy chain comprises an immunologically inert constant region.
17. The protein of embodiment 2 or 3, wherein the heavy chain comprises a wild-type human IgG1 constant region, a human IgG1 constant region comprising the amino acid substitutions L234A, L235A and G237A, a wild-type human IgG2 constant region, a wild-type human IgG4 constant region, or a human IgG4 constant region comprising the amino acid substitution S228P, wherein numbering is according to the EU index as in Kabat.
18. An immunoconjugate comprising the protein of any one of embodiments 1-17, linked to a therapeutic agent.
19. The immunoconjugate of embodiment 18, wherein the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.
20. A pharmaceutical composition comprising the protein of any one of embodiments 1-17 or the immunoconjugate of embodiment 18 or 19, and a pharmaceutically acceptable carrier, diluent or excipient.
21. A nucleic acid molecule encoding
   (a) the heavy chain amino acid sequence;
   (b) the light chain amino acid sequence; or
   (c) both the heavy chain and the light chain amino acid sequences of the protein of any one of embodiments 1-17.
22. An expression vector comprising the nucleic acid molecule of embodiment 21.
23. A recombinant host cell comprising the nucleic acid molecule of embodiment 21 or the expression vector of embodiment 22.
24. A method of producing a protein, the method comprising:
   culturing a recombinant host cell comprising the expression vector of embodiment 22 under conditions whereby the nucleic acid molecule is expressed, thereby producing the protein; and isolating the protein from the host cell or culture.
25. A method for enhancing an anti-cancer immune response in a subject, the method comprising administering to the subject a therapeutically effective amount of the protein of any one of embodiments 1-17, the immunoconjugate of embodiment 18 or 19, or the pharmaceutical composition of embodiment 20.
26. A method for treating cancer in a subject, the method comprising administering to the subject a therapeutically effective amount of the protein of any one of embodiments 1-17, the immunoconjugate of embodiment 18 or 19, or the pharmaceutical composition of embodiment 20.
27. A method for ameliorating a symptom of cancer in a subject, the method comprising administering to the subject a therapeutically effective amount of the protein of any one of embodiments 1-17, the immunoconjugate of embodiment 18 or 19, or the pharmaceutical composition of embodiment 20.
28. The method of any one of embodiments 25-27, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.
29. A protein comprising a first heavy chain and a first light chain,
   wherein the first heavy chain comprises:
      (a) a first portion of a first antigen binding domain (P1), comprising a first heavy chain variable domain (VH), and optionally a first heavy chain constant domain 1 (CH1);
      (b) a first linker, wherein the first linker is cleavable by a protease; and
      (c) a first portion of a second antigen binding domain (P2), comprising a second VH and optionally a second CH1; wherein:
         (i) the N-terminus of the first linker is linked to the C-terminus of the first CH1 of P1 and the C-terminus of the first linker is linked to the N-terminus of the second VH of P2; or
         (ii) the N-terminus of the first linker is linked to the C-terminus of the first VH of P1 and the C-terminus of the first linker is linked to the N-terminus of the second VH of P2; and
      wherein the first light chain comprises:
      (a) a second portion of the first antigen binding domain (P3), comprising a first light chain variable domain (VL), and optionally a first light chain constant domain (CL);
      (b) a second linker, wherein the second linker is cleavable by a protease; and
      (d) a second portion of the second antigen binding domain (P4), comprising a second VL, and optionally a second CL; wherein:
         (i) the N-terminus of the second linker is linked to the C-terminus of the first CL of P3 and the C-terminus of the second linker is linked to the N-terminus of the second VL of P4; or
         (ii) the N-terminus of the first linker is linked to the C-terminus of the first VL of P3 and the C-terminus of the second linker is linked to the N-terminus of the second VL of P4; and
      wherein the first antigen binding domain binds to PD-L1; and
      wherein the when the first linker and the second linker are cleaved, the second antigen binding domain binds to CD89.
30. The protein of embodiment 29, wherein the CH1 of P1 is covalently linked to the CL of P3.
31. The protein of embodiment 29 or 30, wherein the CH1 of P2 is covalently linked to the CL of P4.
32. A protein comprising a first heavy chain and a first light chain, wherein the first heavy chain comprises:
   (a) a first portion of a first antigen binding domain (P1), comprising a first heavy chain variable domain (VH);
   (b) a first linker, wherein the first linker is cleavable by a protease; and
   (c) a first portion of a second antigen binding domain (P2), comprising a second VH;
wherein the N-terminus of the first linker is linked to the C-terminus of P1, and the C-terminus of the first linker is linked to the N-terminus of the second VH of P2; wherein the first light chain comprises:
   (c) a second portion of the first antigen binding domain (P3), comprising a first light chain variable domain (VL);
   (d) a second linker, wherein the second linker is cleavable by a protease; and
   (d) a second portion of the second antigen binding domain (P4), comprising a second VL;
   wherein the N-terminus of the second linker is linked to the C-terminus of P3, and the C-terminus of the second linker is linked to the N-terminus of the second VL of P4;
   wherein the first antigen binding domain binds to PD-L1; and
   wherein the when the first linker and the second linker are cleaved, the second antigen binding domain binds to CD89.
33. The protein of embodiment 32, wherein the P1 further comprises a first heavy chain constant domain 1 (CH1), wherein the C-terminus of the first CH1 of P1 is linked to the N-terminus of the first linker.
34. The protein of embodiment 33, wherein the N-terminus of the first CH1 of P1 is linked to the C-terminus of the VH of P1.
35. The protein of embodiment 32, wherein the P3 further comprises a first CL, wherein the C-terminus of the first CL of P3 is linked to the N-terminus of the second liker.
36. The protein of embodiment 35, wherein the N-terminus of the first CL of P3 is linked to the C-terminus of the first VL of P3.
37. The protein of any one of embodiments 32-36, wherein the P2 further comprises a second CH1, wherein the N-terminus of the second CH1 of P2 is linked to the C-terminus of the second VH of P2.
38. The protein of any one of embodiments 32-37, wherein the P4 further comprises a second CL, wherein the N-terminus of the second CL of P4 is linked to the C-terminus of the second VL of P4.
39. The protein of any one of embodiments 35-38, wherein the first CH1 of P1 is covalently linked to the first CL of P3.
40. The protein of any one of embodiments 35-39, wherein the second CH1 of P2 is covalently linked to the second CL of P4.
41. The protein of any one of embodiments 29-40, wherein the first antigen binding domain comprises P1 and P3.
42. The protein of any one of embodiments 29-41, wherein the P1 and the P3 are covalently linked to form a Fab.
43. The protein of any one of embodiments 29-42, wherein the second antigen binding domain comprises P2 and P4.
44. The protein of any one of embodiments 29-43, wherein the P2 and the P4 are covalently linked to form a Fab.
45. The protein of any one of embodiments 29-44, wherein the first heavy chain further comprises a first dimerization domain.
46. The protein of embodiment 45, wherein the first dimerization domain comprises a first hinge domain and a first immunoglobulin constant domain (Fc domain).
47. The protein of embodiment 46, wherein the Fc domain comprises a first heavy chain constant domain 2 (CH2) and a first heavy chain constant domain 3 (CH3).
48. The protein of embodiments 45-47, wherein the C-terminus of the P2 is linked to the N-terminus of the first hinge domain.
49. The protein of any one of embodiments 29-48, wherein the first linker comprises an amino acid sequence having at least 85% identity to any one of amino acid sequence of SEQ ID NO: 1, SEQ ID NO: 2, SEQ ID NO: 3, or SEQ ID NO: 4.
50. The protein of embodiment 49, wherein the first linker comprises an amino acid sequence of SEQ ID NO: 1, SEQ ID NO: 2, SEQ ID NO: 3, or SEQ ID NO: 4.
51. The protein of any one of embodiments 29-50, wherein the second linker comprises an amino acid sequence having at least 85% identity to any one of amino acid sequence of SEQ ID NO: 1, SEQ ID NO: 2, SEQ ID NO: 3, or SEQ ID NO: 4.
52. The protein of embodiment 51, wherein the second linker comprises an amino acid sequence of SEQ ID NO: 1, SEQ ID NO: 2, SEQ ID NO: 3, or SEQ ID NO: 4.
53. The protein of any one of embodiments 29-52, wherein the first VH of P1 comprises:
   (a) a heavy chain complementarity determining region 1 (HCDR1) amino acid sequence of SEQ ID NO: 12, SEQ ID NO: 34, or SEQ ID NO: 36;
   (b) a heavy chain complementarity determining region 2 (HCDR2) amino acid sequence of SEQ ID NO: 13, SEQ ID NO: 35, or SEQ ID NO: 37; and
   (c) and a heavy chain complementarity determining region 3 (HCDR3) amino acid sequence of SEQ ID NO: 14 or SEQ ID NO: 38.
54. The protein of any one of embodiments 29-53, wherein the first VL of P3 comprises:
   (a) a light chain complementarity determining region 1 (LCDR1) amino acid sequence of SEQ ID NO: 15 or SEQ ID NO: 39;
   (b) a light chain variable domain comprising a light chain complementarity determining region 1 (LCDR2) amino acid sequence of SEQ ID NO: 16 or DA; and
   (c) a light chain variable domain comprising a light chain complementarity determining region 3 (LCDR3) amino acid sequence of SEQ ID NO: 17.
55. The protein of any one of embodiments 29-54, wherein the second VH of P2 comprises:
   (a) a HCDR1 amino acid sequence of SEQ ID NO: 18, SEQ ID NO: 40, or SEQ ID NO: 21;
   (b) a HCDR2 amino acid sequence of SEQ ID NO: 19, SEQ ID NO: 41, or SEQ ID NO: 43; and
   (c) a HCDR3 amino acid sequence of SEQ ID NO: 20 or SEQ ID NO: 44.
56. The protein of any one of embodiments 29-55, wherein the second VL of P4 comprises:
   (a) a LCDR1 amino acid sequence of SEQ ID NO: 21 or SEQ ID NO: 45;
   (b) a LCDR2 amino acid sequence of SEQ ID NO: 22 or GA; and
   (c) a LCDR3 amino acid sequence of SEQ ID NO: 23.
57. The protein of any one of embodiments 29-56, wherein the first VH of P1 comprises a sequence having at least 75%, 80%, 85%, 90%, 95%, or 99% sequence identity to the sequence of SEQ ID NO: 24.
58. The protein of any one of embodiments 29-57, wherein the first VH of P1 comprises the sequence of SEQ ID NO: 24.
59. The protein of any one of embodiments 29-58, wherein the first VL of P3 comprises a sequence having at least 75%, 80%, 85%, 90%, 95%, or 99% sequence identity to the sequence of SEQ ID NO: 25.
60. The protein of any one of embodiments 29-59, wherein the first VL of P3 comprises a sequence of SEQ ID NO: 25.
61. The protein of any one of embodiments 29-60, wherein the second VH of P2 comprises a sequence having at least 75%, 80%, 85%, 90%, 95%, or 99% sequence identity to the sequence of SEQ ID NO: 26.
62. The protein of any one of embodiments 29-61, wherein the second VH of P2 comprises the sequence of SEQ ID NO: 26.
63. The protein of any one of embodiments 29-62, wherein the second VL of P4 comprises a sequence having at least 75%, 80%, 85%, 90%, 95%, or 99% sequence identity to the sequence of SEQ ID NO: 27.
64. The protein of any one of embodiments 29-63, wherein the second VL of P4 comprises the sequence of SEQ ID NO: 27.
65. The protein of any one of embodiments 29-64, wherein the first heavy chain comprises:
   (a) the first VH of P1 having at least 75%, 80%, 85%, 90%, 95%, or 99% sequence identity to the sequence of SEQ ID NO: 24; and
   (b) the second VH of P2 having at least 75%, 80%, 85%, 90%, 95%, or 99% sequence identity to the sequence of SEQ ID NO: 26.
66. The protein of any one of embodiments 29-65, wherein the first heavy chain comprises:
   (a) the first VH of P1 comprising the sequence of SEQ ID NO: 24; and
   (b) the second VH of P2 comprising the sequence of SEQ ID NO: 26.
67. The protein of any one of embodiments 29-66, wherein the first light chain comprises:
   (a) the first VL of P3 having at least 75%, 80%, 85%, 90%, 95%, or 99% sequence identity to the sequence of SEQ ID NO: 25; and
   (b) the second VL of P4 having at least 75%, 80%, 85%, 90%, 95%, or 99% sequence identity to the sequence of SEQ ID NO: 27.
68. The protein of any one of embodiments 29-67, wherein the first light chain comprises:
   (a) the first VL of P3 comprising the sequence of SEQ ID NO: 25; and
   (b) the second VL of P4 comprising the sequence of SEQ ID NO: 27.
69. The protein of any one of embodiments 29-68,
   (a) wherein the first heavy chain comprises:
      (i) the first VH of P1 having at least 75%, 80%, 85%, 90%, 95%, or 99% sequence identity to the sequence of SEQ ID NO: 24; and
      (ii) the second VH of P2 having at least 75%, 80%, 85%, 90%, 95%, or 99% sequence identity to the sequence of SEQ ID NO: 26; and
   (b) wherein the first light chain comprises:
      (i) the first VL of P3 having at least 75%, 80%, 85%, 90%, 95%, or 99% sequence identity to the sequence of SEQ ID NO: 25; and
      (ii) the second VL of P4 having at least 75%, 80%, 85%, 90%, 95%, or 99% sequence identity to the sequence of SEQ ID NO: 27.
70. The protein of any one of embodiments 29-69,
   (a) wherein the first heavy chain comprises:
      (i) the first VH of P1 comprising the sequence of SEQ ID NO: 24; and
      (ii) the second VH of P2 comprising the sequence of SEQ ID NO: 26; and
   (b) wherein the first light chain comprises:
      (i) the first VL of P3 comprising the sequence of SEQ ID NO: 25; and
      (ii) the second VL of P4 comprising the sequence of SEQ ID NO: 27.
71. The protein of any one of embodiments 29-70, wherein the first CH1 of P1 comprises a sequence having at least 75%, 80%, 85%, 90%, 95%, or 99% sequence identity to the sequence of SEQ ID NO: 5.
72. The protein of any one of embodiments 29-71, wherein the first CH1 of P1 comprises the sequence of SEQ ID NO: 5.
73. The protein of any one of embodiments 29-72, wherein the second CH1 of P2 comprises a sequence having at least 75%, 80%, 85%, 90%, 95%, or 99% sequence identity to the sequence of SEQ ID NO: 5.
74. The protein of any one of embodiments 29-73, wherein the second CH1 of P2 comprises the sequence of SEQ ID NO: 5.
75. The protein of any one of embodiments 29-74, wherein the first CL of P3 is a kappa light chain constant domain.
76. The protein of embodiment 75, wherein the kappa light chain constant domain comprises a sequence having at least 75%, 80%, 85%, 90%, 95%, or 99% sequence identity to the sequence of SEQ ID NO: 7.
77. The protein of embodiment 75 or 76, wherein the kappa light chain constant domain comprises the sequence of SEQ ID NO: 7.
78. The protein of any one of embodiments 29-77, wherein the second CL of P4 is kappa light chain constant domain.
79. The protein of embodiment 78, wherein the kappa light chain constant domain comprises a sequence having at least 75%, 80%, 85%, 90%, 95%, or 99% sequence identity to the sequence of SEQ ID NO: 7.
80. The protein of embodiment 78 or 79, wherein the kappa light chain constant domain comprises the sequence of SEQ ID NO: 7.
81. The protein of any one of embodiments 29-80, wherein the first heavy chain comprises a sequence having at least 75%, 80%, 85%, 90%, 95%, or 99% sequence identity to the sequence of SEQ ID NO: 30 or SEQ ID NO: 31.
82. The protein of any one of embodiments 29-81, wherein the first heavy chain comprises the sequence of SEQ ID NO: 30 or SEQ ID NO: 31.
83. The protein of any one of embodiments 29-82, wherein the first light chain comprises a sequence having at least 75%, 80%, 85%, 90%, 95%, or 99% sequence identity to the sequence of SEQ ID NO: 28 or SEQ ID NO: 29.
84. The protein of any one of embodiments 29-83, wherein the first light chain comprises the sequence of SEQ ID NO: 28 or SEQ ID NO: 29.
85. The protein of any one of embodiments 29-84, wherein the first heavy chain and the first light chain comprise the sequence of:
   SEQ ID NO: 30 and SEQ ID NO: 28, respectively;
   SEQ ID NO: 31 and SEQ ID NO: 28, respectively;
   SEQ ID NO: 30 and SEQ ID NO: 29, respectively; or
   SEQ ID NO: 31 and SEQ ID NO: 29, respectively.
86. The protein of any one of embodiments 46-85, wherein the first dimerization domain comprises a sequence having at least 75%, 80%, 85%, 90%, 95%, or 99% sequence identity to the sequence of SEQ ID NO: 32.
87. The protein of any one of embodiments 46-86, wherein the first dimerization domain comprises the sequence of SEQ ID NO: 32.
88. The protein of any one of embodiments 29-87, wherein the first heavy chain comprises one or more heavy chain constant region selected from the group consisting of IgG constant region or functional fragment thereof, IgE constant region or functional fragment thereof, IgM constant region or functional fragment thereof, IgD constant region or functional fragment thereof, IgA constant region or functional fragment thereof, IgY constant region or functional fragment thereof, IgG1 constant region or functional fragment thereof, IgG2 constant region or functional fragment thereof, IgG3 constant region or functional fragment thereof, IgG4 constant region or functional fragment thereof, IgA1 constant region or functional fragment thereof, and IgA2 constant region or functional fragment thereof.
89. The protein of any one of embodiments 29-88, wherein the first heavy chain comprises one or more immunologically inert constant region.
90. The protein of any one of embodiments 29-89, wherein the first heavy chain comprises one or more heavy chain constant region selected from the group consisting of wild-type human IgG1 constant region, a human IgG1 constant region comprising the amino acid substitutions L234A, L235A and G237A, a wild-type human IgG2 constant region, a wild-type human IgG4 constant region, and a human IgG4 constant region comprising the amino acid substitution S228P, wherein numbering is according to the EU index as in Kabat.
91. The protein of embodiment 89 or 90, wherein the heavy chain constant region comprises a sequence having at least 75%, 80%, 85%, 90%, 95%, or 99%, or 100% sequence identity to the sequence of SEQ ID NO: 6, SEQ ID NO: 8, SEQ ID NO: 9, SEQ ID NO: 10, SEQ ID NO: 11.
92. The protein of any one of embodiments 29-91, further comprising a second heavy chain.
93. The protein of embodiment 92, wherein the second heavy chain further comprises a second dimerization domain.
94. The protein of embodiment 93, wherein the second dimerization domain comprises a sequence having at least 75%, 80%, 85%, 90%, 95%, or 99% sequence identity to the sequence of SEQ ID NO: 32.
95. The protein of any embodiment 93 or 94, wherein the second dimerization domain comprises the sequence of SEQ ID NO: 32.
96. The protein of embodiment 93, wherein the first dimerization domain and the second dimerization domain are dimerized.
97. The protein of any one of embodiments 100-104, wherein the second heavy chain comprises:
   (a) a second P1;
   (b) a third linker, wherein the first linker is cleavable by a protease; and
   (c) a second P2.
98. The protein of embodiment 97, further comprising a second light chain, wherein the second light chain comprises:
   (a) a second P3;
   (b) a fourth linker; and
   (c) a second P4.
99. The protein of any preceding embodiment, wherein the C-terminus of the first heavy chain is linked to the N-terminus of the first dimerization domain and the C-terminus of the first light chain is linked to the N-terminus of the second dimerization domain.
100. A protein comprising the structure of: [P1]-[A]-[P2]-[P3]-[B]-[P4], wherein:
   (a) [P1] comprises a first heavy chain variable domain (VH) and optionally a first heavy chain constant domain 1 (CH1) of a first antigen binding domain;
   (b) [A] comprises a first linker, wherein the first linker is cleavable by a protease;
   (c) [P2] comprises a second VH and optionally a second CH1 of a second antigen binding domain;
   (d) [P3] comprises a first light chain variable domain (VL) and optionally a first light chain constant domain (CL) of the first antigen binding domain;
   (e) [B] comprises a second linker, wherein the second linker is cleavable by a protease;
   (f) [P4] comprises a second VL and optionally a second CL of the second antigen binding domain;
   (g) [P1] and [P3] are covalently linked;
   (h) [P2] and [P4] are covalently linked;
   (i) C-terminus of [P1] is linked to N-terminus of [A];
   (j) C-terminus of [A] is linked to N-terminus of [P2];
   (k) C-terminus of [P3] is linked to N-terminus of [B];
   (l) C-terminus of [B] is linked to N-terminus of [P4];
   wherein the first antigen binding domain binds to PD-L1;
   wherein when [A] and [B] are cleaved, the second antigen binding domain binds to CD89.

What is claimed is:

1. A protein comprising a first polypeptide chain comprising a heavy chain and a second polypeptide chain comprising a light chain,
wherein the heavy chain comprises, in N-terminus to C-terminus order, an anti-PD-L1 heavy chain variable (VH) domain, a first CH1 domain, a first linker, an anti-CD89 VH domain, and a second CH1 domain;
wherein the light chain comprises, in N-terminus to C-terminus order, an anti-PD-L1 light chain variable (VL) domain, a first immunoglobulin light chain constant region, a second linker, an anti-CD89 VL domain, and a second immunoglobulin light chain constant region.

2. The protein of claim 1, wherein the heavy chain comprises in N-terminus to C-terminus order, the anti-PD-L1 VH domain, the first CH1 domain, the first linker, the anti-CD89 VH domain, the second CH1 domain, a hinge, a CH2 domain, and a CH3 domain.

3. The protein of claim 1, wherein the protein further comprises a third polypeptide chain comprising a hinge and a Fc region, wherein the third polypeptide comprises the amino acid sequence of SEQ ID NO: 32.

4. (canceled)

5. The protein of claim 1, wherein the first linker comprises the amino acid sequence of SEQ ID NO: 1, SEQ ID NO: 2, SEQ ID NO: 3, or SEQ ID NO: 4.

6. The protein of claim 1, wherein the second linker comprises the amino acid sequence of SEQ ID NO: 1, SEQ ID NO: 2, SEQ ID NO: 3, or SEQ ID NO: 4.

7. The protein of claim 1,
wherein the anti-PD-L1 VH domain comprises a HCDR1 comprising the amino acid sequence of SEQ ID NO: 12, a HCDR2 comprising the amino acid sequence of SEQ ID NO: 13, and a HCDR3 comprising the amino acid sequence of SEQ ID NO: 14;
wherein the anti-PD-L1 VL domain comprises a LCDR1 comprising the amino acid sequence of SEQ ID NO: 15, a LCDR2 comprising the amino acid sequence of SEQ ID NO: 16, and a LCDR3 comprising the amino acid sequence of SEQ ID NO: 17;
wherein the anti-CD89 VH domain comprises a HCDR1 comprising the amino acid sequence of SEQ ID NO: 18, a HCDR2 comprising the amino acid sequence of SEQ ID NO: 19, and a HCDR3 comprising the amino acid sequence of SEQ ID NO: 20; and
wherein the anti-CD89 VL domain comprises a LCDR1 comprising the amino acid sequence of SEQ ID NO: 21, a LCDR2 comprising the amino acid sequence of SEQ ID NO: 22, and a LCDR3 comprising the amino acid sequence of SEQ ID NO: 23.

8. The protein of claim 1, wherein the anti-PD-L1 VH domain comprises the amino acid sequence of SEQ ID NO: 24, and the anti-PD-L1 VL domain comprises the amino acid sequence of SEQ ID NO: 25.

9. The protein of claim 1, wherein the anti-CD89 VH domain comprises the amino acid sequence of SEQ ID NO: 26, and the anti-CD89 VL domain comprises the amino acid sequence of SEQ ID NO: 27.

10. (canceled)

11. The protein of claim 1, wherein the heavy chain comprises the amino acid sequence of SEQ ID NO: 30 or SEQ ID NO: 31.

12. The protein of claim 1, wherein the light chain comprises the amino acid sequence of SEQ ID NO: 28 or SEQ ID NO: 29.

13. The protein of claim 3, wherein
(a) the heavy chain comprises the amino acid sequence of SEQ ID NO: 30 and the light chain comprises the amino acid sequence of SEQ ID NO: 28; or
(b) the heavy chain comprises the amino acid sequence of SEQ ID NO: 31 the light chain comprises the amino acid sequence of SEQ ID NO: 29.

14.-17. (canceled)

18. An immunoconjugate comprising the protein of claim 1, linked to a therapeutic agent.

19. (canceled)

20. A pharmaceutical composition comprising the protein of claim 1, and a pharmaceutically acceptable carrier, diluent or excipient.

21. A nucleic acid molecule encoding
(a) the heavy chain amino acid sequence;
(b) the light chain amino acid sequence; or
(c) both the heavy chain and the light chain amino acid sequences of the protein of claim 3.

22.-25. (canceled)

26. A method for treating cancer in a subject, the method comprising administering to the subject a therapeutically effective amount of the protein of claim 1.

27. (canceled)

28. The method of claim 26, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, oropharyngeal cancer, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

29. The protein of claim 1, wherein the anti-PD-L1 VH domain comprises an amino acid sequence having at least 90% sequence identity to the sequence of SEQ ID NO: 24, and the anti-PD-L1 VL domain comprises an amino acid sequence having at least 90% sequence identity to the sequence of SEQ ID NO: 25.

30. The protein of claim 1, wherein the anti-CD89 VH domain comprises an amino acid sequence having at least 90% sequence identity to the sequence of SEQ ID NO: 26, and the anti-CD89 VL domain comprises an amino acid sequence having at least 90% sequence identity to the sequence of SEQ ID NO: 27.

31. The protein of claim 1, wherein the heavy chain comprises an amino acid sequence having at least 90% sequence identity to the sequence of SEQ ID NO: 30 or SEQ ID NO: 31.

32. The protein of claim 1, wherein the light chain comprises an amino acid sequence having at least 90% sequence identity to the sequence of SEQ ID NO: 28 or SEQ ID NO: 29.
